(19) United States
(12) Patent Application Publication
(10) Pub. No.: US 2016/0206262 A1
(43) Pub. Date: Jul. 21, 2016

(54) EXTENDED VOLUME IMAGING

(71) Applicant: GENERAL ELECTRIC COMPANY, SCHENECTADY, NY (US)

(72) Inventors: David Allen Langan, Clifton Park, NY (US); Bernhard Erich Hermann Claus, Niskayuna, NY (US); Hao Lai, Niskayuna, NY (US); Gary Michael Idelchik, Saratoga Springs, NY (US)

(21) Appl. No.: 14/602,051

(22) Filed: Jan. 21, 2015

(51) Int. Cl. A61B 6/00; A61B 6/04; A61B 6/02

(52) U.S. Cl. CPC A61B 6/481; A61B 6/025; A61B 6/5205; A61B 6/0407; A61B 6/0457

(57) ABSTRACT

The present disclosure relates to the acquisition of image data over an extended field of view using an interventional tomosynthesis system. In one embodiment, the interventional tomosynthesis system has a base offset from the longitudinal axis of a patient table, such that movement of the table relative to the imager may be performed during tomosynthesis projection acquisition. One or both of the imager and the table may move to accomplish such relative motion.

### BACKGROUND

The subject matter disclosed herein relates to tomosynthesis imaging and, in particular, to tomosynthesis imaging over an extended imaging extent.

Various medical procedures involve the insertion and navigation of a tool within a patient's body. For example, needle-based procedures (e.g., lung biopsy, vertebroplasty, RF ablation of liver tumors, and so forth) may involve the insertion and navigation of a needle or needle associated tool through the body of a patient. Such procedures are guided and, therefore, benefit from the acquisition and display of real-time imaging data to assist in the navigation process. For example, such image data may be used to safely guide the device to the target while avoiding critical structures (e.g., arteries and veins) and obstructions (e.g., bones).

As part of such procedures it may, therefore, be desirable to acquire images over a range of the patient's anatomy in excess over what is typically imaged based on the size or extent of the detector arrangement. For example, it may be desirable to image the vasculature of the patient over an extended extent, such as in a digital subtraction angiography (DSA) procedure, to facilitate the interventional guidance. In some instances, for example, a bolus of contrast may be injected into a patient and observed by radiographic imaging as it travels through the vasculature, rendering the vasculature radio-opaque when present in sufficient concentration. Such contrast-enhanced images may then undergo a subtraction procedure whereby a contemporaneous non-contrast enhanced image (i.e., a mask image) is digitally subtracted from the contrast-enhanced image to generate an image of the contrast enhanced vascular tree without other structures being shown.

In some instances it may be desirable to follow or “chase” the bolus along the anatomy, i.e., over an extended anatomic extent, so as to obtain a greater amount of contrast image data where the anatomy of interest exceeds the field of view of the detector. Such bolus chasing procedures are conventionally done using two-dimensional (2D) imaging techniques, where radiographic images are obtained from a single view angle relative to the patient but along a Z-axis relative to the length patient. That is, the imaging apparatus does not rotate about the patient, but may move along the length of the patient to follow a contrast bolus through the vasculature. In this manner a contrast bolus injected into the iliac artery and may be chased or followed through the leg/peripheral vasculature.

Such two-dimensional imaging techniques, though useful, do not provide the type of three-dimensional (3D) vasculature information that may be desirable for interventional procedures. In particular, vasculature that overlaps within the projection may not be visually distinguished and the three-dimensional geometry of the vasculature may remain unclear. Further, 2D imaging provides limited opportunity to quantify the vascular geometry, which is inherently three-dimensional. However, those imaging modalities that are suitable for 3D DSA with bolus chase, such as computed tomography (CT) and magnetic resonance (MR) imaging, are generally unsuitable for interventional procedures due to the lack of patient access and/or due to the inability to easily use tools in the imaging environment of such systems, such as due to the magnetic fields or high radiation dose levels.

Another imaging modality, C-arm CBCT, also allows acquisition of 3D imaging data but is generally not suitable for use in acquiring image data over an extended anatomic extent of the patient, such as may be used in tool guidance or a bolus chase procedure. In particular, C-arm CBCT systems typically rotate the imaging apparatus about the patient and may, therefore, not be suitable for bolus chase procedures due to the imager and spin geometry relative to the patient, which may be suitable for imaging only the anatomy at the center of the spin, and/or medical personnel. Similarly, with C-arm CBCT the time required to perform a single volumetric image acquisition and the time delay between consecutive acquisitions may be prohibitive for bolus chase and guidance type procedures.

### BRIEF DESCRIPTION

In one embodiment, an imaging method is provided. In accordance with this imaging method, an X-ray source and an X-ray detector of a tomographic imaging system are moved within a limited angular range along an orbital path with respect to an imaged volume. The X-ray source is constrained to move on a first side of the imaged volume and the X-ray detector is constrained to move on a second side of the imaged volume opposite the first side. The X-ray source and the X-ray detector are moved relative to a patient table one or two dimensions concurrent with and in addition to the orbital path motion. A projection dataset is acquired using the X-ray source and the X-ray detector while moving the X-ray source and the X-ray detector in the orbital path and relative to the patient table. One or more three-dimensional images having an extended field of view are generated using the first projection dataset.

In a further embodiment, an imaging system is provided. In accordance with one embodiment, the imaging system includes an X-ray source constrained to move on a first side of a patient support, an X-ray detector constrained to move on a second side of the patient support opposite the first side, and one or more support structures configured to support the X-ray source and the X-ray detector. In some embodiments, the support structure may be mobile, for example, an automated guided vehicle, while in other embodiments the support structure may be mounted to a floor, a ceiling, or a wall of an examination room. Alternatively, the X-ray source and detector may be on independent support structures, for example robotic arms. The support structures are offset to a longitudinal axis of the patient support so as to allow motion of the support structures and the patient support relative to one another. The imaging system also includes a controller and one or more processing components configured, alone or in combination, to: operate the X-ray source and X-ray detector during an image acquisition so as to acquire a set of projections; move the X-ray source and the X-ray detector on their respective sides of the patient support during the image acquisition; move one or both of the patient support or the support structures during the image acquisition relative to one another; and reconstruct the set of projections to generate one or more three-dimensional images having an extended field of view.

In an additional embodiment, an imaging method is provided. In accordance with this imaging method, a support arm is moved relative to a side of a patient table. The support arm connects to a C-arm supporting an X-ray source configured to move on a first side of the patient table and an X-ray detector configured to move on a second side of the patient table opposite the first side. During a first acquisition, a mask set of projections is acquired while moving the X-ray detector and the X-ray source at least longitudinally along the side of the patient table. A contrast bolus is administered to a patient. During a second acquisition, a contrast set of projections is acquired while moving the X-ray detector and the X-ray source at least longitudinally along the side of the patient table. A volumetric reconstruction of vasculature of the patient is generated using the mask set of projections and the contrast set of projections. The mask and contrast projections may be: 1) taken at the same location, thereby enabling subtraction in projection domain; or 2) taken at different locations, with subtraction occurring in the image domain.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical view of a single-plane imaging system for use in producing images in accordance with aspects of the present disclosure;

FIG. 2 is a diagrammatical view of a bi-plane imaging system for use in producing images in accordance with aspects of the present disclosure;

FIG. 3 is a schematic front view of a single-plane imaging system in which an imaging apparatus obtains projection data along a plane via rotation about two axes, in accordance with aspects of the present disclosure;

FIG. 4 depicts movement of a source and detector of a single-plane C-arm tomosynthesis system configured to perform a bolus chase procedure, in accordance with aspects of the present disclosure;

FIG. 5 depicts a conventional cone beam computed tomography system configured for a spin acquisition;

FIG. 6 depicts the system of FIG. 4 in conjunction with table motion, in accordance with aspects of the present disclosure; and

FIG. 7 depicts a bi-plane C-arm tomosynthesis system configured to perform a bolus chase procedure, in accordance with aspects of the present disclosure.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

In certain navigational procedures, such as a catheterization procedure, it is useful to be able to visualize an extended anatomic extent of the patient or to be able to image off-center anatomic locations. For example, being able to image over an extended anatomic extent may be useful in imaging the vasculature of a patient as part of the procedure. As part of such vascular imaging, it may be desirable to utilize bolus chase procedures, where the imaging apparatus can be moved relative to the patient so as to acquire contrast-enhanced image data as the bolus travels through the patient's body. Such bolus chase approaches may be implemented in 2D imaging schemes, but are not feasible using conventional C-arm CBCT approaches due to the rotation of the imaging apparatus conflicting with translation of the patient relative to the imager over the patient, and the acquisition time for a single volume.

The present approach addresses certain of these issues and provides for an extent of a patient volume to be imaged beyond what would typically be viewable with stationary C-arm mounted components. By way of example, in one implementation this enhanced imaging extent may be leveraged to allow or improve upon a bolus chase procedure using 3D digital subtraction angiography and an interventional C-arm, as discussed in greater detail herein. However, in a broader context, the present approach provides an enhanced or increased imaging extent by allowing relative motion of the patient table or support with respect to the imager along one dimension (i.e., longitudinally or along the long axis of the patient and table) or more than one dimension (i.e., longitudinally and laterally), regardless of whether contrast enhancement or digital subtraction is employed. Certain implementations utilize a C-arm tomosynthesis system employing a 2-axis (or more than 2-axis) tomography acquisition motion (which may be continuous) over a limited angular range. In conjunction with this tomosynthesis acquisition motion, the relative motion of the patient relative to the imager may be implemented, such as by moving one or both of the patient support (i.e., table) or one or more supports (e.g., an L-arm or independent robotic arms) of the imaging system which support the imager components. In this manner, the motion of the tomosynthesis acquisition is performed in such a way so as to allow table and/or support relative motion, which allows the imaging system to acquired images over an extended anatomic extent, such as to move relative to a bolus and thereby facilitate a bolus chase procedure. In such implementations, the table motion may undergo one-dimensional or two-dimensional translational motion that extends the 3D field of view over the anatomy of interest. Though digital subtraction, such as DSA, is one procedure that may benefit from extension of the imaged volume, as discussed herein, the presently disclosed approaches may also be useful in contexts other than digital subtraction in which a greater anatomic extent of the patient needs to be visualized.

As discussed herein, aspects of the present approach may utilize tomosynthesis as part of an interventional or other imaging procedure. In certain embodiments, the X-ray detector and source (e.g., an X-ray tube) continuously orbit within a plane, respectively above and below the patient support table. This allows motion of the patient relative to the orbiting detector and source. Further, in this arrangement, access to the patient is significantly improved relative to computed tomography (CT) imaging system or conventional C-arm Cone Beam Computed Tomography (CBCT) imaging system as the source and detector are not rotated or spun about the axis of the patient. For example, as discussed in greater detail below, CBCT is performed using an L-arm positioned at the head of the table, severely limiting table motion.

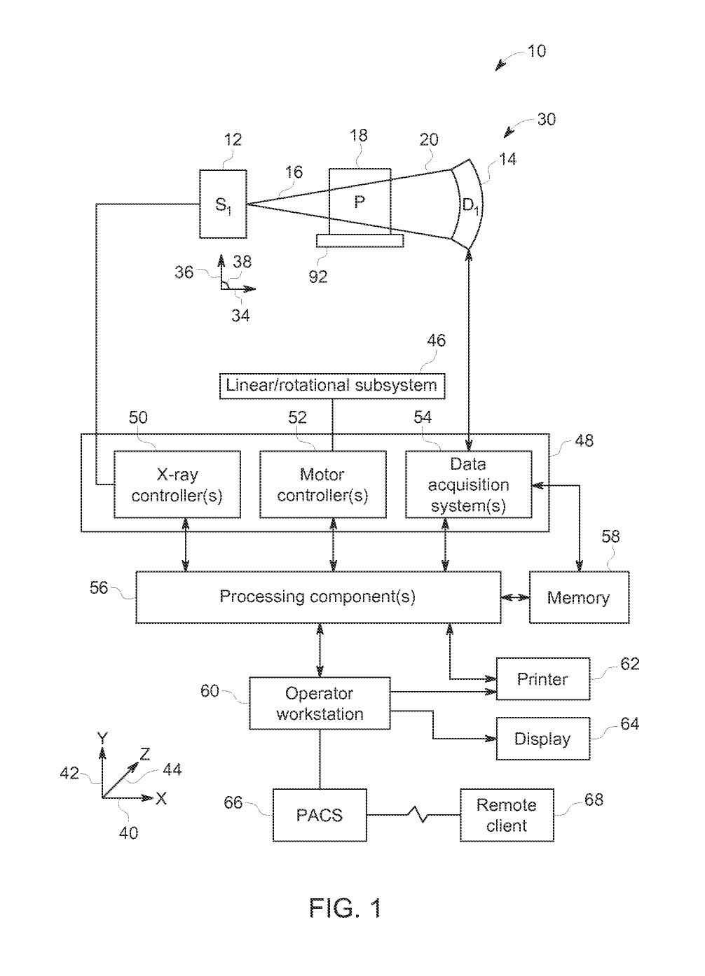

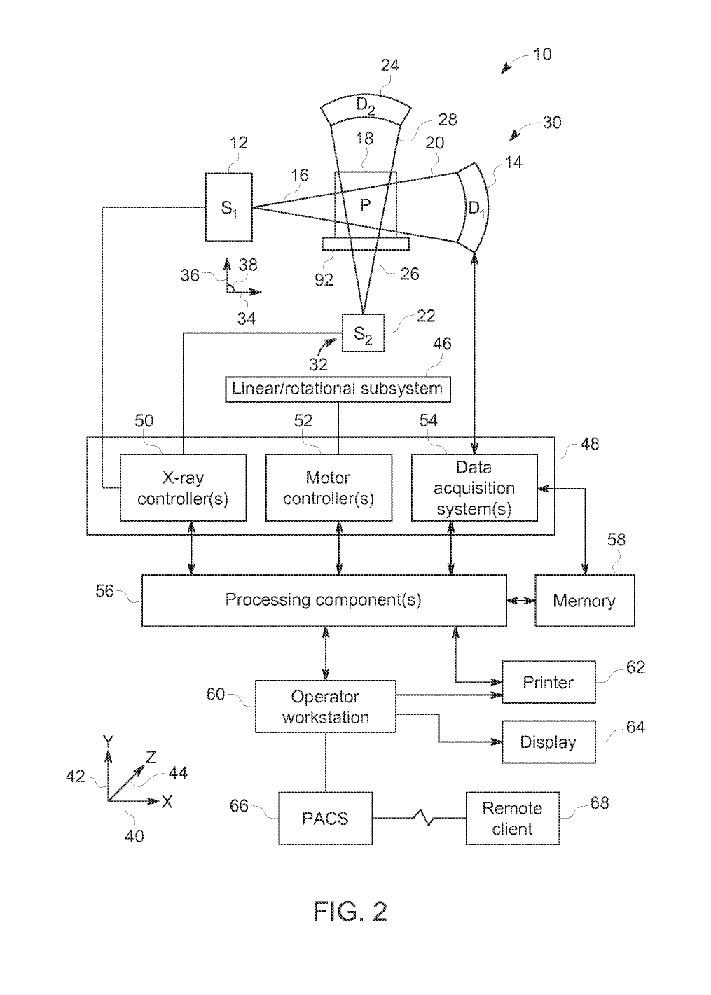

With respect to the imaging system, either a single-plane or a bi-plane tomosynthesis system may be used to implement the present approach. Turning to FIGS. 1 and 2, an example, of both single plane (FIG. 1) and bi-plane (FIG. 2) imaging systems 10 are depicted. Both tomosynthesis imaging systems 10 are designed to acquire X-ray attenuation data at a variety of views around a patient and suitable for navigational imaging. In the embodiment illustrated in FIG. 1, imaging system 10 includes a first source of X-ray radiation 12 and a first detector 14. The first X-ray source 12 may be an X-ray tube, a distributed X-ray source (such as a solid-state or thermionic X-ray source) or any other source of X-ray radiation suitable for the acquisition of medical or other images. In certain implementations, the X-ray source 12 may be switchable between different emission profiles (e.g., profiles having different mean energy), such as to facilitate dual-energy imaging protocols.

The X-rays 16 generated by the first source 12 pass into a region in which a patient 18 is positioned during a procedure. In the depicted example, the X-rays 16 are collimated to be a cone-shaped beam, e.g., a cone-beam, which passes through the imaged volume. A portion of the X-ray radiation 20 passes through or around the patient 18 (or other subject of interest) and impacts a detector array, represented generally as the first detector 14. Detector elements of the first detector 14 produce electrical signals that represent the intensity of the incident X-rays 20. These signals are acquired and processed to reconstruct images of the features within the patient 18.

In the present example, the first source 12 and first detector 14 may be a part of a first imager 30. The first imager 30 may acquire X-ray images or X-ray projection data over a limited angular range with respect to one side or facing (e.g., the anterior/posterior (AP) direction) of the patient 18, thereby defining data in a first plane (e.g., a frontal plane of the patient 18). In this context, an imaging plane may be defined as a set of projection directions that are located within a certain angular range relative to a reference direction. For example, the frontal imaging plane may be used to describe projection views within an angular range that is within, for example, 60 degrees of the PA (posterior/anterior) direction of the patient. Similarly, a lateral imaging plane may be described as the set of projection directions within an angular range that is within 60 degrees of the lateral/horizontal left/right projection direction.

As depicted, the first imager 30 positions the first source 12 and the first detector 14, at rest, generally along a first direction 34, which may correspond to the AP direction of the patient 18 in certain embodiments. The second imager 32, when present (e.g., as shown in FIG. 2), positions a second source 22 and a second detector 24, at rest, generally along a second direction 36, which may correspond to the lateral direction of the patient 18 in certain embodiments.

In accordance with present embodiments, the first imager 30 (and/or the second imager 32 when present) may be moved relative to the patient or imaged object and relative to one another along one or more axes, such as the Z-axis running lengthwise (i.e., longitudinally) through the patient 18, during an examination procedure during which projection data is acquired. For example, the first imager 30 may move about a first axis of rotation 40, a second axis of rotation 42, or a third axis of rotation 44, or any combination thereof, and the second imager 32, when present, may move about any one or a combination of these axes as well.

The movement of the first imager 30 (and/or the second imager 32) may be initiated and/or controlled by one or more linear/rotational subsystems 46. In addition, in certain embodiments, the patient table 92 may be moved linearly by the linear/rotational subsystems 46 in conjunction with, or instead of, the imagers. The linear/rotational subsystems 46, as discussed in further detail below, may include support structures, motors, gears, bearings, and the like, that enable the rotational and/or translational movement of the imagers and/or patient table 92. In one embodiment, the linear/rotational subsystems 46 may include a first structural apparatus (e.g., a C-arm apparatus having rotational movement about at least two axes) supporting the first source and detector 12, 14, and/or an arm or arms supporting the table 92.

A system controller 48 may govern the linear/rotational subsystems 46 that initiate and/or control the movement of one or more of the first imager 30, the second imager 32, and patient table 92. In practice, the system controller 48 may incorporate one or more processing devices that include or communicate with tangible, non-transitory, machine readable media collectively storing instructions executable by the one or more processors to perform the operations described herein, including the coordinated movement of the table 92 and imagers 30, 32. The system controller 48 may also include features that control the timing of the activation of the X-ray sources, for example, to control the acquisition of X-ray attenuation data obtained during a particular imaging sequence. The system controller 48 may also execute various signal processing and filtration functions, such as for initial adjustment of dynamic ranges, interleaving of digital projection data, and so forth. Therefore, in general, the system controller 48 may be considered to command operation of the imaging system 10 to execute examination protocols. It should be noted that, to facilitate discussion, reference is made below to the system controller 48 as being the unit that controls acquisitions, movements, and so forth, using the imagers. However, embodiments where the system controller 48 acts in conjunction with other control devices (e.g., other control circuitry local to the imagers or remote to the system 10) are also encompassed by the present disclosure.

In the present context, the system controller 48 includes signal processing circuitry and various other circuitry that enables the system controller 48 to control the operation of the imagers and the linear/rotational subsystems 46. In the illustrated embodiment, the circuitry may include an X-ray controller 50 configured to operate the X-ray sources so as to time the operations of these sources and to interleave the acquisition of X-ray attenuation data when needed. Circuitry of the system controller 48 may also include one or more motor controllers 52. The motor controllers 52 may control the activation of various components that are responsible for moving the X-ray sources, the detectors, and/or the table 92. For example, the motor controllers 52 may coordinate movement of the first imager 30 (and the second imager 32 when present) such that the imagers obtain data from different projection directions, maintain a desired degree of angular separation, and also for collision avoidance. In other words, the motor controllers may implement a particular trajectory for one or both imagers. Likewise, the motor controllers 52 may coordinate linear motion of the table 92 (along one- or two-dimensions) with one or more of the imaging trajectories of the imagers.

The system controller 48 is also illustrated as including one or more data acquisition systems 54. Generally, the detectors may be coupled to the system controller 48, and more particularly to the data acquisition systems 54. The data acquisition systems 54 may receive data collected by read out electronics of the detectors, and in certain embodiments may process the data (e.g., by converting analog to digital signals or to perform other filtering, transformation, or similar operations).

It should be noted that the tangible, non-transitory, machine-readable media and the processors that are configured to perform the instructions stored on this media that are present in the system 10 may be shared between the various components of the system controller 48 or other components of the system 10. For instance, as illustrated, the X-ray controller 50, the motor controller 52, and the data acquisition systems 54 may share one or more processing components 56 that are each specifically configured to cooperate with one or more memory devices 58 storing instructions that, when executed by the processing components 56, perform the image acquisition techniques described herein. Further, the processing components 56 and the memory components 58 may coordinate in order to perform various image acquisition and/or reconstruction processes.

The system controller 48 and the various circuitry that it includes, as well as the processing and memory components 56, 58, may be accessed or otherwise controlled by an operator via an operator workstation 60. The operator workstation 60 may include any application-specific or general-purpose computer that may include one or more programs (for example one or more imaging programs) capable of enabling operator input for the techniques described herein. The operator workstation 60 may include various input devices such as a mouse, a keyboard, a trackball, or any other similar feature that enables the operator to interact with the computer. The operator workstation 60 may enable the operator to control various imaging parameters, for example, by adjusting certain instructions stored on the memory devices 58.

The operator workstation 60 may be communicatively coupled to a printer 62 for printing images, patient data, and the like. The operator workstation 60 may also be in communication with a display 64 that enables the operator to view various parameters in real time, to view images produced by the acquired data, and the like. The operator workstation 60 may also, in certain embodiments, be communicatively coupled to a picture archiving and communication system (PACS) 66. Such a system may enable the storage of patient data, patient images, image acquisition parameters, and the like. This stored information may be shared throughout the imaging facility and may also be shared with other facilities, for example, a remote client 68. The remote client 68 may include hospitals, doctors' offices, or any other similar client.

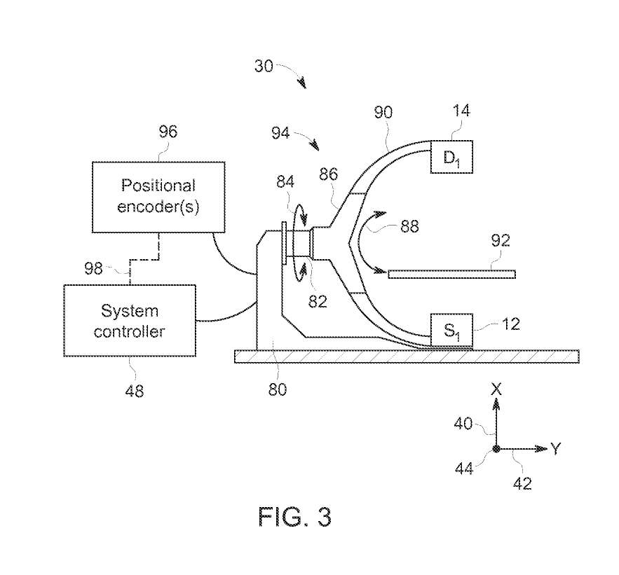

Various aspects of the present approaches may be further appreciated with respect to FIG. 3, which is a view of an embodiment of single-plane tomosynthesis imaging system as discussed herein and shown with respect to FIG. 1. In the depicted example, the system gantry and table 92 are seen from a “head-on” perspective in which the head (or feet) of the patient, if present on the table 92, would be closest to the viewer. As illustrated, the system includes a first imager 30. As will be appreciated, and as discussed with respect to FIG. 2, a second imager 32 may also be present, though such a second imager is not shown in FIG. 3 so as to simplify illustration and discussion.

The first imager 30, as illustrated, includes a first base 80 and a rotatable extension 82 extending from the first base 80, which may be a mobile base, and forming an L-arm. In the illustrated embodiment, the L-arm formed by the first base 80 and extension 82 is floor-mounted. In other embodiments, however, the L-arm may instead be mounted to the ceiling or a wall of the scan room. The L-arm may also be incorporated into an automated vehicle (such as the Discovery IGS 730, available from General Electric Company) such that the imaging apparatus is portable, as opposed to being fixed within a single examination room. As discussed in certain embodiments, the L-arm may be movable, either manually or via a motorized controller 52 of the imaging system. As such, the L-arm may be configured to move the first imager 30 and the associated C-arm with respect to the table 92, such as in the Z-direction (or in the Z-direction and a perpendicular direction) with respect to table 92. It should be appreciated that the second imager 32, when present may be similarly configured.

Turning back to FIG. 3, the rotatable extension 82 is depicted as extending generally along a second axis of rotation 42, and enables the first source 12 and the first detector 14 to move about the second axis of rotation 42. For example, the rotatable extension 82 may enable the first source 12 and the first detector 14 to move about the second axis of rotation 42 in a manner that maintains their position relative to one another throughout the rotation. The rotation enabled by the rotatable extension 82 is shown as double-headed arrow 84. The rotatable extension 82 is coupled to a first moving structure 86 (e.g., directly or indirectly via an extension arm), which enables the first source 12 and the first detector 14 to move about the third axis of rotation 44. This rotation about the third axis of rotation 44 is depicted as double-headed arrow 88.

The first moving structure 86 may be a geared or track structure that is motively coupled to a first support structure 90 that physically supports the first source 12 and the first detector 14, and may be in the form of a C-arm, or any other mechanism that positions the first source 12 and the first detector 14 on either side of the patient 18. As illustrated, the first support structure 90 includes an arcuate structure (i.e., a C-arm) that extends from a first side of a patient table 92, around the patient table 92, and to a second side of the patient table 92. In this way, the first source 12 and the first detector 14 generally remain positioned at opposite ends and/or on opposite sides of the patient (not shown) positioned on patient table 92. Together, the first base 80, the rotatable extension 82, the first moving structure 86, and the first support structure 90 may be considered to be the first structure 94 of the first imager 30.

The first imager 30 may include various motors, actuators, or other features responsible for movement of the various structures of the first imager 30, and they may be communicatively coupled to one or more positional encoders 96. One or more positional encoders 96 may encode the respective positions of any one or more components of the first imager 30 in a manner that facilitates processing by the system controller 48. In such an implementation, the positional encoders 96 may provide feedback 98 (for example via wired or wireless signals) to the system controller 48. The system controller 48 may use this feedback 98 to control either or both the first imager 30 or the table 92. Similarly, when present the second imager 32 may have a comparable positional encoder used to generate feedback and assist in the positioning of the second imager.

By way of example, the system controller 48 may simultaneously move the first source 12 and the first detector 14 together about the first axis of rotation 40, the second axis of rotation 42, or the third axis of rotation 44, or any combination thereof, and obtain first X-ray attenuation data for a subset of the traversed view angles. At substantially the same time, the system controller 48 may simultaneously move the second source 22 and the second detector 24, if present, together about the first, second, or third axes of rotation 40, 42, 44, or any combination thereof, in order to obtain second X-ray attenuation data for one or more of the traversed view angles. Similarly, in coordination with the movement of the first and second imagers in this manner, the respective L-arms supporting the imagers and/or the table 92 may be moved in the Z-direction, as discussed herein, to facilitate a bolus chase procedure. In one embodiment, the system controller 48 may receive positional information from the positional encoders (e.g., encoder 96) and may calculate a trajectory (or update a modeled trajectory) for the respective source and detector using this positional feedback information.

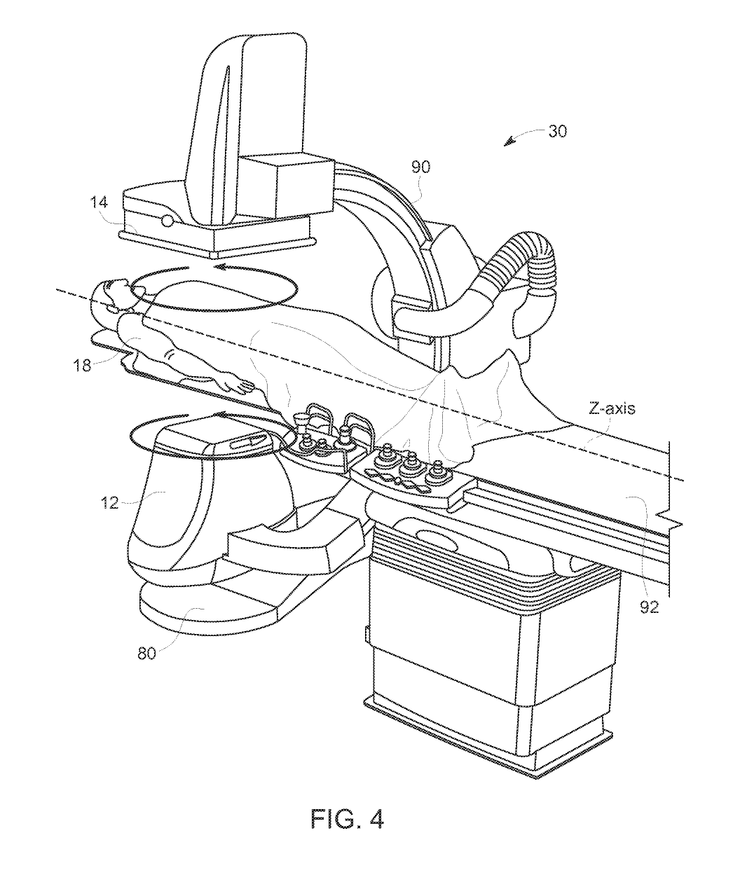

Furthermore, the system controller 48 may synthesize one or more volumetric images (including mask images, contrast images, and digital subtraction images) using data obtained by the first imager 30 and, in some instances, supplemented by the second imager 32 when present. For example, in one embodiment, projection images/data obtained by the second imager 32 may be used to supplement the data obtained by the first imager 30, such as for reconstruction of a 3D mask, contrast, or subtraction image. In such an embodiment, the first imager 30 may perform a first acquisition of data using a first trajectory (e.g., a circular, ellipsoidal, or similar path traced by the first source 12 below the patient 18 and a corresponding circular, ellipsoidal, or similar path traced by the first detector above the patient 18, referred to herein as a frontal tomosynthesis trajectory). An example of such a motion (i.e., an “orbit” as used herein) is conceptually demonstrated in FIG. 4 in the context of a first imager 30. In this example, the first imager 30 may obtain projection data from a plurality of projection directions, but these projection directions may be limited by the angular range of motion of the first imager 30 (e.g., the limited angular displacement about the second rotational axis 42) and/or the presence of structures associated with the second imager 32, or other devices or structures. In one embodiment, the angular range of the trajectory may also be limited due to temporal constraints. In one example, the angular range of an elliptical orbit that is part of the trajectory may be defined by the requirement that the orbit may have to be traversed in a certain amount of time, e.g., in 3 seconds or less.

While the preceding discussion with respect to FIGS. 1, 3, and 4 relates primarily to a single plane system to simplify and facilitate explanation of basic system configuration, components and terminology, as noted above, a second imager 32 may also be present in certain embodiments. Such an implementation is shown in FIG. 2. In the depicted example, the bi-plane imaging system includes a second source 22 of X-ray radiation and a second detector 24 supported by a second structural apparatus (e.g., a C-arm apparatus) to form the second imager 32. The second source 22 also generates X-rays 26, which may be collimated to form any suitable shape (e.g., a cone) and, in some instances may be switchable between different emission profiles. The X-rays 26 are partially attenuated such that a portion 28 passes through the patient 18 and impacts the second detector 24. The second imager 32 may acquire data within a different limited angular range with respect to a different side or facing (e.g., a lateral direction) of the patient 18, thereby defining data in a second plane (e.g., a lateral plane of the patient 18).

The first and second directions 34, 36 in which the respective imagers are oriented may be oriented at an angle 38 relative to one another. The angle 38 may be any angle that is suitable to enable the first and second imagers 30, 32 to acquire projection data over separate and distinct limited angular ranges with respect to the patient. Further, the angle 38 may be adjusted by various features of the system 10, such as various linear and rotational systems or, in other embodiments, by an operator. Generally, the angle 38 may be between 30 and 180 degrees, but it may be desirable in certain embodiments for the first and second imagers 30, 32 to be oriented crosswise relative to one another, such as between 30 and 90 degrees, or between 90 and 150 degrees. In one embodiment, the angle 38 is approximately 90 degrees. As discussed herein, the rotation of the first and second imagers 30, 32 may be coordinated in accordance with a specified protocol. In a further implementation, the second imager 32 may be stationary and may, therefore, only acquire projection data from a fixed position relative to the first imager 30.

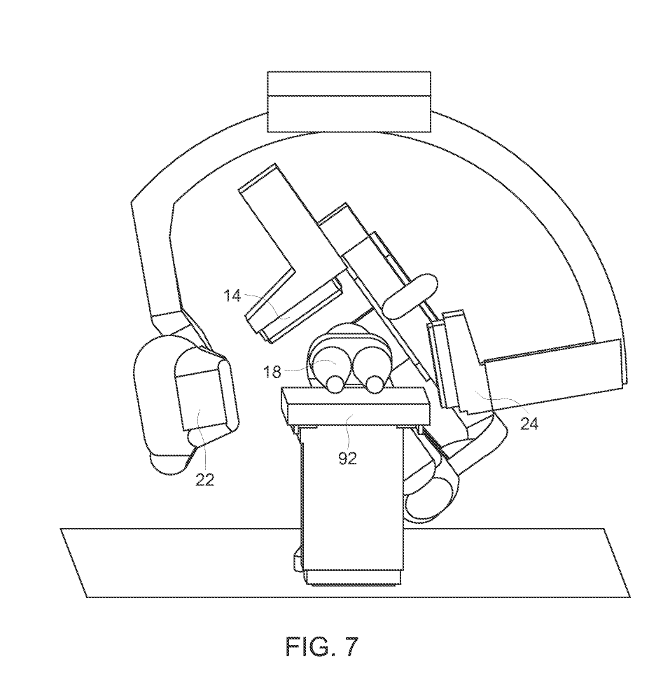

In accordance with certain embodiments, a second imager 32 (shown in FIGS. 2 and 7) may move about the same or a different rotational axis at projection directions or Z-axis positions beyond those obtained by the first imager 30 (e.g., at larger angles relative to the frontal plane of the patient 18 or downstream or upstream along the patient). Thus, the data obtained by a second imager 32, if present, may complement the data obtained by the first imager 30, and may enable the system controller 48 (or other reconstruction device) to perform 3D tomosynthesis reconstruction using a more complete set of data. For example, in one embodiment, this data may be considered to be obtained by the second imager 32 via lateral plane imaging, in that the second X-ray source 22 may generate a trajectory that may trace a line or non-linear path along a lateral direction of the patient 18 (and at angular displacements therefrom). Various tomosynthesis reconstruction algorithms that may be used to reconstruct a 3D volumetric image of the imaged region of interest include those that are well known by those of ordinary skill in the art, and may be of the analytical or iterative type, including but not limited to filtered back projection. In certain embodiments, data acquisition by the first and second imagers 30, 32 may be interleaved in order to avoid signal contamination between the imagers.

With the preceding in mind, as used herein, a tomosynthesis trajectory of an imager may be described as a path (e.g., a line, curve, circle, oval, and so forth, as well as combinations thereof) traced by an X-ray source during image acquisition. A tomosynthesis acquisition by an imager or imager subsystem occurs over a limited angular range with respect to the patient (such as with respect to one side, e.g., the front back, left side, or right side, of the patient), and thus a trajectory will typically move the source within this limited angular range with respect to the imaged subject. Such trajectories may be periodic in that the path traced by the X-ray source may be repeated throughout the examination.

As noted above, and as shown in FIG. 4, each period of motion may be referred to as an orbit. For example, in the context of an oval or circular trajectory, an endpoint of one orbit may correspond to the beginning point of the next orbit. Similarly, linear or non-linear paths traced by the X-ray source may be repeated in a back-and-forth manner, leading to a periodic type trajectory. For example, an X-ray source may be moved (i.e., have a trajectory) in a circular or oval periodic motion (e.g., an orbit) in front of the patient, without rotating around the patient, thereby acquiring X-ray projection data over a limited angular range with respect to the patient. By way of example, the present approach relates to the use of a C-arm to perform tomosynthesis imaging in a navigational or digital subtraction angiography context. In this imaging mode, the detector 14 and tube (e.g., source 12) orbit continuously within a plane above and below the table 92. In one embodiment, the orbit generally has a half tomosynthesis angle of 15° to 30° and an orbit period of 3 to 8 seconds.

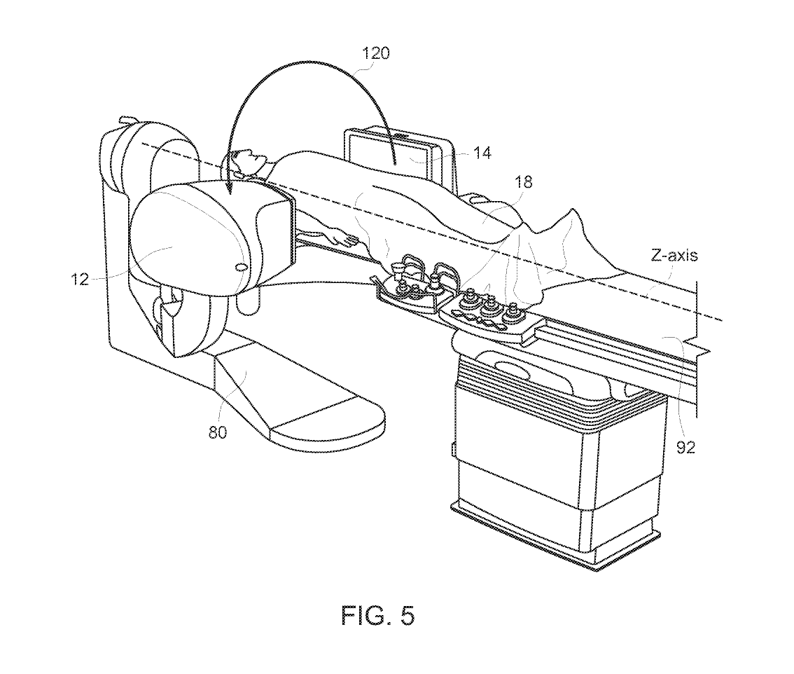

Such a motion is in contrast to the spin-type source motion or trajectory typically associated with C-arm cone-beam computed tomography (CBCT) type systems and acquisitions. By way of example, and turning to FIG. 5, a conventional C-arm CBCT arrangement is shown. In such a conventional arrangement, the base 80 of the L-arm is positioned at the head of the table 92 (i.e., at 0° relative to the table 92 and patient 18), allowing the source 12 and detector 14 to spin (arrow 120) about the patient 18 such that both the source 12 and detector 14 rotate about the front, back, and lateral sides of the patient 18. In this arrangement, neither the table 92 nor the source 12 and detector 14 can be moved relative to one another to an appreciable extent due to the placement of the base 80, which must be at an end of the table 92 to provide the desired spin motion 120. Thus, the depicted conventional CBCT system is incapable of performing a bolus chasing procedure due to the limited motion of the imager relative to the patient in the Z-direction.

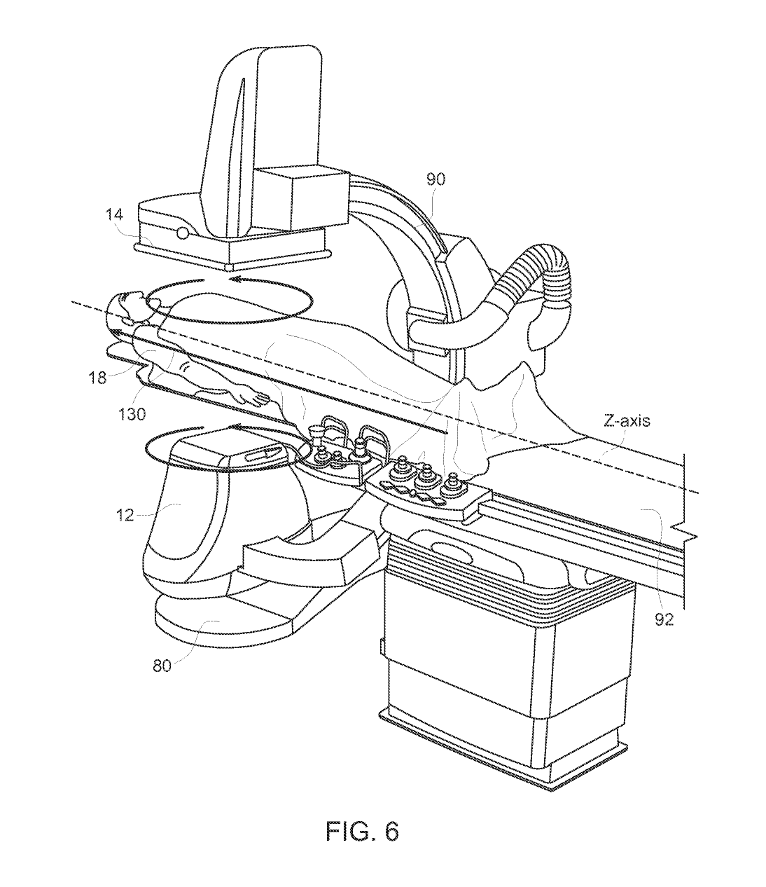

Turning to FIG. 6, the present approach, using a tomosynthesis imaging system as shown in FIG. 4, allows motion of the patient 18 relative to the imager components (e.g., source 12 and detector 14). In particular, tomosynthesis acquisition can be coordinated with one or more of longitudinal and/or lateral motion of the table 92, automatic or manual tracking of bolus progression through the patient 18, or measurements of dynamic physiological processes (e.g., heartbeat and/or respiration) provided by one or more physiological monitors in communication with imaging system. For example, as shown in FIG. 4, in present embodiments the L-arm comprising the base 80 and extension can be at any position, such as positioned at a lateral side of the patient 18 and/or the table 92, and the acquisition trajectory (i.e., orbit) of the imager components adapted. Because the imaging arm and base are not in the path of table motion in the Z-direction, the table 92 and/or imager may move relative to one another in the Z-direction. Consequently tomosynthesis may be employed to perform a 3D bolus chase (or to otherwise acquire projection images over an enhanced or increased longitudinal extent) on an interventional C-arm as the table 92 is moved (arrow 130) relative to the C-arm 90 during the tomosynthesis acquisition in the direction the contrast bolus is moving through the patient.

Likewise, as the imaging base and arm are offset to one lateral side of the patient, and can generally be positioned on either side of the patient, lateral movement of the table 92 relative to the imager is also possible in addition to or instead of longitudinal (i.e., Z-axis) motion. That is, relative motion of the table 92 and imager can be in one or two dimensions. Such lateral motion may be useful for off-center anatomic imaging, to coordinate angular sampling, to gate physiologic events, or to otherwise provide an enhanced lateral imaging extent in addition to the available increased longitudinal extent.

In addition, as noted above, an imaging operation, such as 3D bolus chase, may be performed with a bi-plane system, which may provide improved image quality attributable to the additional projection data. Such an embodiment is shown in FIG. 7, which depicts a view from the foot of table 92. In such an implementation, the posterior-anterior (PA) C-arm (i.e., the first imager 30) would employ an L-arm that is offset from the table 92 in the Z-direction to enable table motion relative to the first imager 90, while the second imager 32 (i.e., the lateral C-arm) would be offset in the Z-direction from the table 92 as well and configured to obtain lateral images of the patient 18. The two C-arm motions (PA and LAT) would be coordinated by system controller 48 to avoid collisions.

As noted above, a bolus chase operation (or other acquisition in which data is acquired over an extended anatomical extent longitudinally and/or laterally) as discussed herein may be realized via table motion and/or L-arm motion. In certain embodiments, the primary purpose of the L-arm is to permit the optimal tomosynthesis trajectory given the performance of the gantry axes and collision constraints. As may be appreciated, the relative motion of the table 92 and the gantry tomosynthesis trajectory influence the angular sampling and field of view over the course of the tomosynthesis acquisition. This relative motion can at least partly be taken into account in the tomosynthesis trajectory configuration and design. Further, the speed associated with the motion of the table and L-arm relative to one another can be varied or coordinated so as to accommodate angular sampling of the tomosynthesis acquisition (e.g., a sinusoidal table speed trajectory) as well as to accommodate patient anatomy and/or dynamic physiological process (e.g., heartbeat and/or respiration). Additionally, the X-ray projection sampling rate may also be varied to achieve the desired angular sampling rate. The relative motion and/or speed may also be accounted for in the reconstruction operation with respect to projection weighting and approximately consistent fields of view over the projections used in the reconstruction operation.

Technical effects of the invention include performing an image acquisition over an extended anatomical extent, such as for a bolus chase operation, using an interventional tomosynthesis system. Further technical effects include generation of digital subtraction images using a C-arm interventional tomosynthesis system having a base or L-arm offset from the longitudinal axis of a patient table, such that movement of the table relative to the imager in one or two dimensions may be performed during tomosynthesis projection acquisition. One or both of the imager and the table may move to accomplish such relative motion.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. An imaging method, comprising:
moving an X-ray source and an X-ray detector of a tomographic imaging system within a limited angular range along an orbital path with respect to an imaged volume, wherein the X-ray source is constrained to move on a first side of the imaged volume and the X-ray detector is constrained to move on a second side of the imaged volume opposite the first side;
moving the X-ray source and the X-ray detector relative to a patient table in one or two dimensions concurrent with and in addition to the orbital path motion;
acquiring a projection dataset using the X-ray source and the X-ray detector while moving the X-ray source and the X-ray detector in the orbital path and relative to the patient table; and
generating one or more three-dimensional images having an extended field of view using the projection dataset.

2. The imaging method of claim 1, wherein the movement of the X-ray source and the X-ray detector along the orbital path and the relative motion of the X-ray source and the X-ray detector with respect to the patient table are coordinated to provide a specified angular sampling.

3. The imaging method of claim 1, wherein a sampling rate of the X-ray source and the X-ray detector is varied to provide a specified angular sampling.

4. The imaging method of claim 1, wherein the X-ray source and the X-ray detector are moved continuously

5. The imaging method of claim 1, wherein the X-ray source is moved in a first two-dimensional plane on the first side of the imaged volume and the X-ray detector is moved in a second two-dimensional plane on the second side of the imaged volume opposite the first side.

6. The imaging method of claim 1, wherein a support structure coupled to the X-ray source and X-ray detector is positioned offset from the longitudinal axis of the patient so as to allow the patient table supporting the patient to move without contacting the support structure.

7. The imaging method of claim 1, wherein a support structure coupled to the X-ray source and X-ray detector is positioned offset from the longitudinal axis of the patient so as to allow the support structure to move relative to the patient table without contacting the patient table.

8. The imaging method of claim 1, wherein moving the X-ray source and the detector relative to the patient table comprises moving one or both the patient table supporting or a support structure supporting the X-ray source and the X-ray detector.

9. The imaging method of claim 1, wherein moving the X-ray source and the X-ray detector relative to the patient table comprises moving X-ray source and the X-ray detector relative to the patient table along one or both of a longitudinal axis associated with the patient table or an perpendicular axis to the longitudinal axis.

10. The imaging method of claim 9, wherein relative movement along one or both of the longitudinal axis or the perpendicular axis is accomplished by moving the patient table.

11. The imaging method of claim 1, wherein movement of the X-ray source and the X-ray detector relative to the patient table in a longitudinal dimension is coordinated so as to do one or both of: improve angular sampling or gate a dynamic physiological process.

12. The imaging method of claim 1, comprising:
administering a contrast bolus to a patient;
wherein the movement of the X-ray source and the X-ray detector relative to the patient table tracks the contrast bolus through the patient;

13. The imaging method of claim 12, wherein the one or more three-dimensional images having the extended field of view comprise one or both of a digital subtraction image generated using a previously acquired mask image or a contrast enhanced image.

14. The imaging method of claim 1, further comprising:
acquiring a concurrent projection dataset using an additional X-ray source and an additional X-ray detector offset from the X-ray source and the X-ray detector along the longitudinal axis; and
reconstructing the concurrent projection dataset in conjunction with the first projection dataset to generate the one or more three dimensional images.

15. An imaging system, comprising:
an X-ray source constrained to move on a first side of a patient support;
an X-ray detector constrained to move on a second side of the patient support opposite the first side;
one or more support structures configured to support the X-ray source and the X-ray detector, wherein the support structures are offset to a longitudinal axis of the patient support so as to allow motion of the support structures and the patient support relative to one another;
a controller and one or more processing components configured, alone or in combination, to:
operate the X-ray source and X-ray detector during an image acquisition so as to acquire a set of projections;
move the X-ray source and the X-ray detector on their respective sides of the patient support during the image acquisition;
move one or both of the patient support or the support structures during the image acquisition relative to one another; and
reconstruct the set of projections to generate one or more three-dimensional images having an extended field of view.

16. The imaging system of claim 15, wherein one or both of the patient support or the support structure are moved during the image acquisition to track a contrast bolus.

17. The imaging system of claim 16, wherein the one or more three-dimensional images comprise one or both of a contrast enhanced image or digital subtraction image generated using a previously acquired mask image and the contrast enhanced image.

18. The imaging system of claim 15, wherein one or both of the motion and speed of the patient support and the support structure relative to one another are coordinated so as to allow one or both of angular sampling or gating of a dynamic physiological process.

19. An imaging method, comprising:
moving a support arm relative to a side of a patient table, wherein the support arm connects to a C-arm supporting an X-ray source configured to move on a first side of the patient table and an X-ray detector configured to move on a second side of the patient table opposite the first side;
during a first acquisition, acquiring a mask set of projections while moving the X-ray detector and the X-ray source at least longitudinally along the side of the patient table;
administering a contrast bolus to a patient;
during a second acquisition, acquiring a contrast set of projections while moving the X-ray detector and the X-ray source at least longitudinally along the side of the patient table; and
generating a volumetric reconstruction of vasculature of the patient using the mask set of projections and the contrast set of projections.

20. The imaging method of claim 19, wherein generating the volumetric reconstruction comprises performing a digital subtraction of the mask set of projections and the contrast set of projections or of respective images generated from the mask set of projections and the contrast set of projections.

21. The imaging method of claim 19, wherein one or both of the motion or speed of the support arm relative to the side of the patient table are coordinated so as to facilitate angular sampling.

22. The imaging method of claim 19, wherein the X-ray source is configured to move in a first two-dimensional plane on the first side of the patient table and the X-ray detector is configured to move in a second two-dimensional plane on the second side of the patient table opposite the first side
